(19) United States
(12) Patent Application Publication
(10) Pub. No.: US 2026/0235541 A1
(43) Pub. Date: Aug. 13, 2026

(54) DEVICE FOR DETECTING FLUORIDE IONS IN A NITRIC MEDIUM

(71) Applicants: COMMISSARIAT A L'ENERGIE ATOMIQUE ET AUX ENERGIES ALTERNATIVES, Paris (FR); ORANO RECYCLAGE, Châtillon (FR)

(72) Inventors: Sophie BOSONNET, Versailles (FR); Olivier CLOUE, Gif-sur-Yvette (FR); Danièle AYRAULT, Saint Germain Les Arpajon (FR); Morgane GUILBERT, Forges les bains (FR); Léo BOSLAND, Chilly-Mazarin (FR); Samuel SUBE, Massy (FR); Kevin GINESTAR, Gif sur Yvette (FR); Laurent JUNOD, La Hague (FR)

(21) Appl. No.: 19/474,789

(22) PCT Filed: Apr. 10, 2024
(86) PCT No.: PCT/EP2024/059758

§ 371 Date: Oct. 13, 2025

(30) Foreign Application Priority Data
Apr. 13, 2023 (FR) FR2303665

(51) Int. Cl. G01N 27/07; G01N 27/04

(52) U.S. Cl. CPC G01N 27/07; G01N 27/045

(57) ABSTRACT

A device for detecting fluoride ions in a nitric medium including a detection probe including: a zirconium measurement element and a zirconium reference element; measurement conductors; an electrical resistance measurement circuit connected to the measurement conductors, and suitable for measuring the relative electrical resistance of a portion of the measurement element, with respect to a portion of the reference element; and a detection circuit suitable for correlating an increase in the relative resistance with the presence of fluoride ions in the nitric medium.

### TECHNICAL FIELD

The invention relates to the field of detection of chemical elements and relates more particularly to the detection of the chemical element fluorine (F) in free form, that is to say in the form of fluoride ions.

### PRIOR ART

It is known to detect the presence of the element fluorine in a solution in particular via reactive tubes, with spectroscopic means, or with chromatographic means.

The current methods and devices are costly to implement and typically require destructive sampling in the solution. Their implementation is in general time-consuming and they are difficult to integrate into a continuous industrial method.

In addition, these methods and devices are generally limited to the detection of the element fluorine overall, whether it is present in a complex or free form, and are not able to distinguish fluoride ions from the element fluorine present in the form of complexes (for example when it is bound to a metal cation, or when it is bound to a proton to form hydrofluoric acid).

### DISCLOSURE OF THE INVENTION

The goal of the invention is to improve the detection devices of the prior art.

To this end, the invention is aimed at a device for detecting fluoride ions in a nitric medium comprising:
a detection probe comprising: a measurement element made of zirconium having a wall defining a closed internal space, this measurement element being intended to be immersed in the nitric medium to be inspected; a reference element made of zirconium, disposed in the internal space of the measurement element and electrically connected by a first end to the measurement element;
measurement conductors electrically connected to the measurement element and to the reference element, and adapted to the measurement of the electrical resistance of a portion of the measurement element and a portion of the reference element;
a circuit for measuring electrical resistance connected to said measurement conductors, and adapted to measure the relative electrical resistance of said portion of the measurement element, relative to said portion of the reference element;
a detection circuit adapted to correlate an increase in said relative resistance with the presence of fluoride ions in the nitric medium.

The invention allows precisely the detection only of fluoride ions in the nitric medium. The device according to the invention can be installed in-line, in a continuous process, to allow continuous monitoring and detection in near real time of the presence of fluoride ions in the nitric medium inspected, without requiring sampling or intervening in the nitric medium.

The device according to the invention can provide a piece of information with high precision, without necessarily resorting to expensive resources.

The invention can be implemented for example in the industries of production of nitric acid, or any method using nitric acid, manufacturing structures intended to receive nitric acid (such as acid storage tanks, or for the treatment of spent nuclear fuel), in the field of manufacturing of chemical monitoring and control instruments, or laboratory chemistry.

The device according to the invention can comprise the following additional features, alone or in combination:
said measurement conductors comprise: a first measurement conductor electrically connected to the measurement element at a first measurement point and a second measurement conductor electrically connected to the measurement element at a second measurement point located at a predetermined distance from the first measurement point;
a first reference measurement conductor electrically connected to the reference element at a first reference measurement point and a second reference measurement conductor electrically connected to the reference element at a second reference measurement point located at a predetermined distance from the first reference measurement point;
the device further comprises a first power supply conductor electrically connected to the measurement element, and a second power supply conductor electrically connected to a second end of the reference element;
said conductors are made of the same material as the measurement element;
the detection probe comprises a body onto which the measurement element is hermetically attached, said conductors being connected at the output of this body to external wiring;
said body is made of the same material as the measurement element;
the measurement element has a tubular shape, and the reference element has an oblong shape extending longitudinally in the measurement element, the reference element being electrically connected to the measurement element at one end of the measurement element onto one end of the measurement element;
the measurement element is formed by a tube closed by a plug onto which the reference element is electrically connected;
the detection probe comprises an electrical insulator material in contact with the inner faces of the measurement element and in contact with the reference element, this electrical insulator material having a thermal conductivity greater than 1.15 W/m. k;
said conductors are welded onto the measurement element by vacuum welding;
the circuit for measuring electrical resistance is adapted to a resistance measurement of the “4-wire” type;
the reference element comes from the same metallurgical casting as the measurement element.

### PRESENTATION OF THE DRAWINGS

Other features and advantages of the invention will emerge from the following non-limiting description, in reference to the appended drawings in which:

FIG. 1 schematically illustrates a device for detecting fluoride ions in a nitric medium according to the invention;

FIG. 2 illustrates in perspective the detection probe of the device of FIG. 1;

FIG. 3 is a cross-sectional view of the detection probe;

FIG. 4 illustrates an example of a relative resistance measurement with the detection probe.

Similar elements shared by the various embodiments bear the same numbers of reference to the figures.

### DETAILED DESCRIPTION

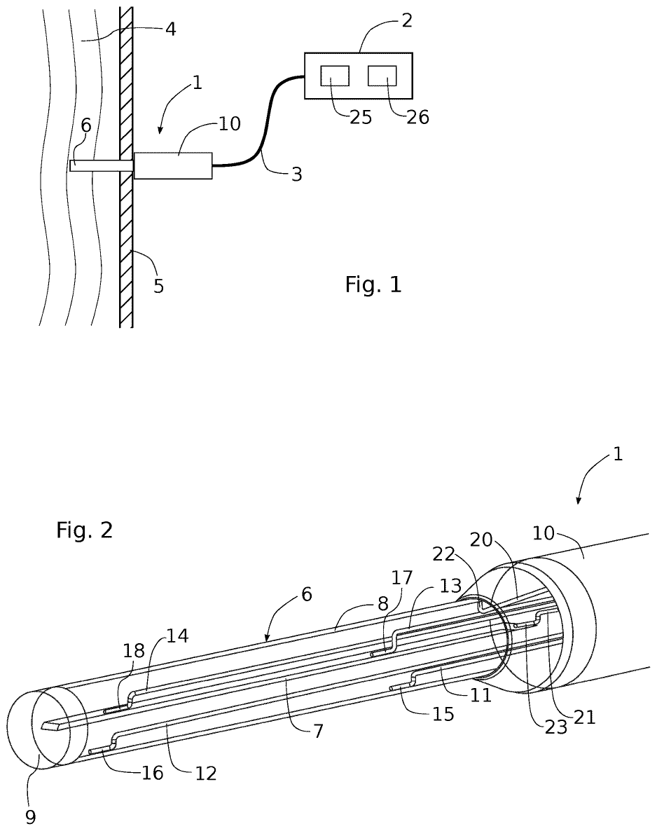

FIG. 1 schematically illustrates a device for detecting fluoride ions in a nitric medium according to the invention.

The detection device comprises a detection probe 1 intended to come in contact with the nitric medium as well as an electronic measurement module 2 comprising the components necessary for the operation of the detection device. The module 2 is connected to the detection probe 1 by one or more cables 3, and comprises the interfaces adapted to the device and in particular to view or export the data and the alerts produced, directly or via additional computer means. The module 2 comprises a circuit 25 for measuring electrical resistance, and a detection circuit 26. The circuits 25, 26 can be formed by any known electronic and/or computer means, with the suitable programming, and optionally by an external computer with a suitable communication interface.

The nitric medium 4 is schematically represented here inside a portion of a container (circulation duct, tank, etc.) which is defined by a wall 5.

The detection probe 1 comprises a measurement element 6 which is a closed envelope adapted to come in contact with the nitric medium 4, with a wall defining a closed inner space, and impermeable to this medium. In this exemplary embodiment, the detection probe 1 is adapted to be mounted in a sealed manner on the wall 5 so that the measurement element 6 is in contact with the nitric medium 4.

The nitric medium 4 can be any solution at least partly comprising nitric acid.

FIG. 2 is a perspective view of the detection probe 1, with the measurement element 6 showed as transparent so as to make its internal components visible.

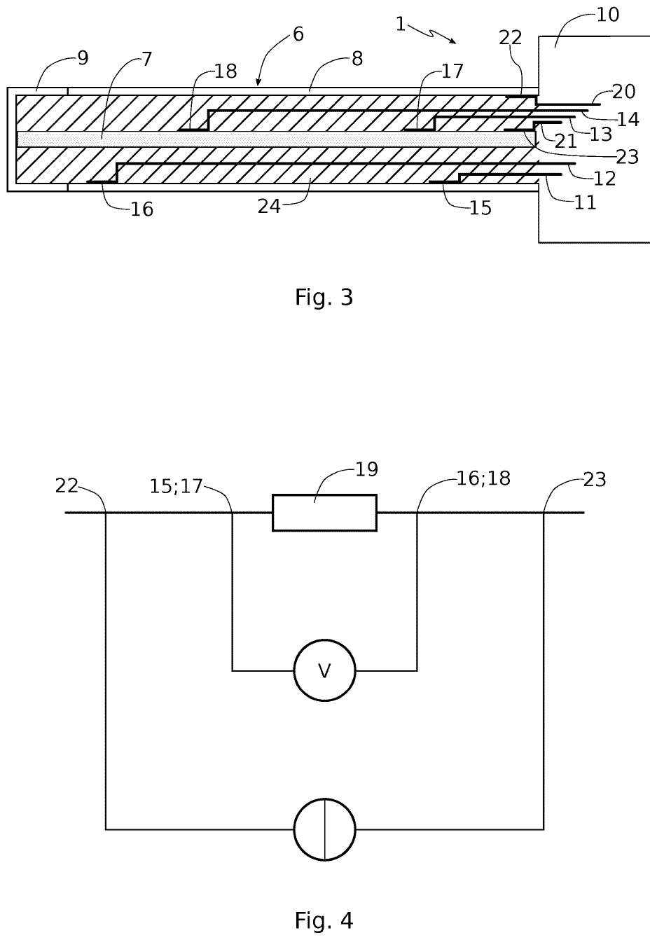

FIG. 3 also shows the detection probe 1, here according to a cross-sectional view. The measurement element 6 is formed by a metal wall made of zirconium.

In reference to FIGS. 2 and 3, the measurement element 6 encloses a reference element 7. The reference element 7 is made of the same material as the measurement element 6. The reference element 7 has an oblong shape extending longitudinally in the measurement element 6. The reference element can consist of any oblong shape: wire, blade or slat, cylinder, etc. In the present example, this oblong shape is a blade.

The reference element 7 is manufactured from the same material and preferably comes from the same metallurgical casting as the measurement element 6, and thus made of zirconium.

In the present example, the measurement element 6 consists of a tube 8 hermetically sealed at its end by a plug 9. The detection probe 1 comprises a body 10 and the measurement element 6 is hermetically coupled onto this body 10, for example by welding.

The reference element 7 is disposed inside the measurement element 6 and is electrically connected to the measurement element 6. In the present example, the reference element 7 is electrically connected to the measurement element 6 at the plug 9, by one of the ends of the reference element 7. In this example, the reference element 7 also extends over the entire length of the measurement element 6.

The measurement element 6 thus has a tubular shape, and the blade which in this example constitutes the reference element 7 extends longitudinally in the measurement element 6, the reference element 7 being electrically connected to the measurement element 6 at one end of said blade onto an end of the tubular shape of the measurement element 6. The electrical connection between the reference element 7 and the measurement element 6 can be carried out by any means that does not impair the sealing of the assembly, such as welding, gluing, etc.

An electrically insulating and thermally conductive material 24 is cast inside the measurement element 6 and maintains the reference element 7 in place, while electrically insulating it from the measurement element 6 but thermally coupling it.

The device also comprises measurement conductors 11, 12, 13, 14 adapted to the measurement:
of the electrical resistance of a portion of the measurement element 6; and
of the electrical resistance, taken as a reference, of a portion of the reference element 7.

Thus, the device here comprises a first measurement conductor 11 electrically connected to the measurement element 6 at a first measurement point 15, as well as a second measurement conductor 12 electrically connected to the measurement element 6 at a second measurement point 16. These measurement points 15, 16 are located on the inner face of the measurement element 6.

Similarly, here the device comprises a first reference measurement conductor 13 electrically connected to the reference element 7 at a first reference measurement point 17, as well as a second reference measurement conductor 14 electrically connected to the reference element 7 at a second reference measurement point 18.

The electrical connection between the measurement conductors 11, 12, 13, 14 at the measurement points 15, 16, 17, 18 is carried out by any known and suitable means such as welding.

The first and second measurement points 15, 16 of the measurement element 6, on the one hand, and the first and second reference measurement points 17, 18 of the reference element 7, on the other hand, are respectively spaced apart by a predetermined distance, defining a portion of the measurement element 6 and of the reference element 7 on which the corresponding electrical resistance is measured.

The measurement of the electrical resistance between each pair of measurement points 15, 16; 17, 18 can be carried out by any known means. In the present example, this measurement of electrical resistance is carried out by a “4-wire” measurement.

For this implementation, the device comprises a first power supply conductor 20 which is electrically connected to the base of the measurement element 6, on its inner face, at a first power supply point 22. The first supply point 22 is located in this case at the contact between the first power supply conductor 20 and the inner face of the measurement element 6. Alternatively, the first power supply conductor 20 can be electrically connected onto the body 10, preferably near the junction between the body 10 and the measurement element 6 (the first power supply point 22 thus being located at the junction between the body 10 and the measurement element 6). This alternative allows a simplification of the method for manufacturing the device.

The device also comprises a second power supply conductor 21 which is electrically connected to the reference element 7 at a second power supply point 23. The second power supply point 23 is located on an end of the reference element 7 which is opposite to the end by which the reference element is electrically connected to the measurement element 6. Thus, the power supply conductors allow to make a current pass in series through the measurement element 6 and the reference element 7.

The measurement conductors 11, 12, 13, 14 and the power supply conductors 20, 21 are connected at the output of the body 10 to external wiring (schematically shown by the cable 3 in FIG. 1) necessary for the connection with the module 2. The conductors 11, 12, 13, 14, 20, 21 are for example sheathed by a suitable electrical insulator polymer.

FIG. 4 schematically illustrates the 4-wire resistance measurement applied to both the measurement element 6 and the reference element 7. In FIG. 4, the resistor 19 schematically represents the resistance to be measured of the portion of measurement element 6 or of reference element 7 located between the two measurement points 15, 16 or 17, 18.

In a known manner for a 4-wire resistance measurement, a current source (contained in the module 2, in the resistance measurement circuit 25) circulates a constant current between the two power supply points 22, 23. This current passes through the measurement element 6 and the reference element 7. The voltage between the two measurement points 15, 16 on the one hand and 17, 18 on the other hand is thus measured accurately (via voltage measurement means contained in the module 2, in the resistance measurement circuit 25) in order to deduce therefrom the value of the resistance 19 by Ohm's law. An accurate measurement of the electrical resistance of the portion of the measurement element 6 and of the portion of the reference element 7 in question is thus carried out continuously, so as to detect minute variations in resistance.

The module 2, via the measurement circuit 25, also carries out a relative measurement of the resistance of said portion of the measurement element 6 with respect to the resistance of said portion of the reference element 7. In other words, the measurement circuit 25 determines the variation in electrical resistance of the portion of the measurement element 6 that goes beyond the variation in the electrical resistance of the portion of the reference element 7. This relative measurement can be carried out by an individual measurement of the resistance of said portion of the measurement element 6, and of said portion of the reference element 7, and the ratio of these voltages is determined by the module 2, optionally with computer means connected to the module 2 by a suitable interface. To obtain this relative resistance, the value of the electrical resistance relative to the reference element 7 can be continuously subtracted from the value of the electrical resistance relative to the measurement element 6.

The measurement of this relative electrical resistance allows to quantify the value of the electrical resistance (of said portion of the measurement element) independently of the temperature. Since the insulating material 24 is thermally conductive, the measurement element 6 and the reference element 7 are subjected substantially to the same temperature, and the variations in the electrical resistance relative to changes in temperature are undergone by both the measurement element 6 and the reference element 7. These variations in electrical resistance due to the temperature are not therefore taken into account in the measurement of said relative electrical resistance, the latter thus only varying under the influence of the interactions of the measurement element 6 with the nitric medium 4.

When the detection device is in service, the measurement element 6 is immersed in the nitric medium 4 and the fluoride ions that are present in the nitric medium react with the material forming the measurement element 6. Thus, the presence of fluoride ions in the nitric medium 4 results in a decrease in thickness of the measurement element 6, which is in contact with the nitric medium 4, and has no impact on the reference element 7, which is protected inside the measurement element 6.

The presence of fluoride ions in the nitric medium results in a decrease (even small) in the thickness of zirconium of the measurement element 6, and this presence is detected by the increase in said relative electrical resistance beyond a predetermined threshold. The detection circuit 26 of the module 2 is adapted (by construction or programming) to correlate an increase in the value of said relative resistance with the presence of fluoride ions in the nitric medium 4. The detection circuit 26 is adapted to detect the presence of fluoride ions in the nitric medium, according to an increase in said relative electrical resistance. The detection circuit 26 can be calibrated for example experimentally or by calculation.

In a particularly advantageous embodiment, the measurement conductors 11, 12, 13, 14, the power supply conductors 20, 21, and the body 10 are made of the same material as the measurement element 6 (made of zirconium). Thus, the entirety of the detection probe 1 can be created by a welding technique such as high-energy-density welding methods like vacuum electron beam or resistance welding, which allow both the assembly of the tube 8 and the plug 9 and the welding of the conductors at the measuring points 15, 16, 17, 18 and at the supply points 22, 23.

The module 2 can be programmed according to any suitable method, for example to continuously verify the variation in said relative resistance and thus qualify the evolution of the presence of fluoride ions in the nitric medium 4.

Alternative embodiments of the device are possible, for example the shape and the arrangements of the measurement element 6 or the means for measuring the relative resistance can be different.

What is claimed is:

1. A device for detecting fluoride ions in a nitric medium, the device comprising:
a detection probe comprising: a measurement element made of zirconium having a wall defining a closed inner space, this measurement element configured to be immersed in the nitric medium to be inspected; a reference element ) made of zirconium, disposed in the inner space of the measurement element and electrically connected by a first end to the measurement element;
measurement conductors electrically connected to the measurement element and to the reference element, and adapted to the measurement of the electrical resistance of a portion of the measurement element and a portion of the reference element;
a circuit for measuring electrical resistance connected to said measurement conductors, and adapted to measure the relative electrical resistance of said portion of the measurement element, relative to said portion of the reference element;
a detection circuit adapted to correlate an increase in said relative resistance with the presence of fluoride ions in the nitric medium.

2. The device according to claim 1, wherein said measurement conductors comprise:
a first measurement conductor electrically connected to the measurement element at a first measurement point and a second measurement conductor electrically connected to the measurement element at a second measurement point located at a predetermined distance from the first measurement point;
a first reference measurement conductor electrically connected to the reference element at a first reference measurement point and a second reference measurement conductor electrically connected to the reference element at a second reference measurement point located at a predetermined distance from the first reference measurement point.

3. The device according to claim 1, further comprising a first power supply conductor electrically connected to the measurement element and a second power supply conductor

(21) electrically connected to a second end of the reference element.

4. The device according to claim 1, wherein said conductors are made of the same material as the measurement element.

5. The device according to claim 1, wherein the detection probe comprises a body onto which the measurement element is hermetically attached, said conductors being connected at the output of this body to external wiring.

6. The device according to claim 5, wherein said body is made of the same material as the measurement element

7. The device according to claim 1, wherein the measurement element has a tubular shape, and the reference element has an oblong shape extending longitudinally in the measurement element, the reference element being electrically connected to the measurement element at one end of the reference element onto one end of the measurement element.

8. The device according to claim 1, wherein the measurement element is formed by a tube closed by a plug onto which the reference element is electrically connected.

9. The device according to claim 1, wherein the detection probe comprises an electrical insulator material in contact with the inner faces of the measurement element and in contact with the reference element, this electrical insulator material having a thermal conductivity greater than 1.15 W/m.k.

10. The device according to claim 1, wherein said conductors are welded onto the measurement element by vacuum welding.

11. The device according to claim 1, wherein the circuit for measuring electrical resistance is adapted to a resistance measurement of the “4-wire” type.

12. The device according to one claim 1, wherein the reference element comes from the same metallurgical casting as the measurement element.
